(19) United States
(12) Patent Application Publication
(10) Pub. No.: US 2003/0153891 A1
(43) Pub. Date: Aug. 14, 2003

(54) Multifunctional disposal tape on an absorbent article

(76) Inventor: Kenneth John Molee, Suwanee, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 10/074,214

(22) Filed: Feb. 14, 2002

(51) Int. Cl. A61F 13/15

(52) U.S. Cl. 604/385.13; 604/389

(57) ABSTRACT

The present invention relates generally to an absorbent article having a multifunctional disposal fastening mechanism disposed on the outer cover thereof. The absorbent article has an improved disposal fastening mechanism that provides for a convenient and simple disposal of the article, while at the same time providing relevant or pleasing indicia on the multifunctional disposal fastening mechanism. The multifunctional disposal fastening mechanism is capable of maintaining the garment in a rolled up configuration, and it includes visible indicia.

### FIELD OF THE INVENTION

The present invention relates generally to an absorbent article having a multifunctional disposal fastening mechanism disposed on the outer cover thereof. The absorbent article has an improved disposal fastening mechanism that provides for a convenient and simple disposal of the article, while at the same time providing relevant or pleasing indicia on the multifunctional disposal fastening mechanism.

### BACKGROUND OF THE INVENTION

It is known to use adhesive tape fastening elements to fasten the rear waist region of an absorbent diaper to the front waist region to thereby place the diaper on a wearer. Various adhesive fastening mechanisms are disclosed in, for example, U.S. Reissue Pat. No. 26,151, and U.S. Pat. Nos. 3,848,594, and 4,963,140, the disclosures of which are incorporated by reference herein in their entirety. These adhesive tape fastening systems provide a secure mechanism for fitting and maintaining the absorbent diaper on a wearer. To the extent that the adhesive tape fastening systems are re-fastenable, some of the known systems permit the disposable absorbent diaper to be rolled up into a tight package for disposal. After rolling, the rolled article can be secured in the rolled up configuration and then disposed of.

Other re-fastenable mechanisms include the conventional hook-and-loop fasteners. These mechanical fasteners typically are capable of being fastened and re-fastened more frequently than tape fasteners. The mechanical hook-and-loop fasteners can be used to re-fasten the disposable absorbent diaper after it is rolled up if a receiving surface is disposed on the outer surface of the garment. Such a configuration is disclosed in U.S. Pat. No. 4,963,140. These disposal systems all make use of the mechanical fasteners (adhesive tapes, or hook-and-loop systems) that are used in absorbent diaper to attach one waist region to another after donning the garment on the wearer.

The present day absorbent diapers are being replaced to an ever greater extent with disposable pants-type diapers, or so-called training pants, for slightly older diaper-wearing children or for adults. Pants-type diapers have a number of desirable features. For example, pants-type diapers resemble conventional underpants and, thereby, can help somewhat older diaper-wearing children to learn to perform the operations required to put on conventional underpants as well as encourage the child to graduate to underpants. However, present pants-type garments can be difficult to put on, especially by young children.

Disposable pants-type diapers, and adult incontinence absorbent garments typically are not equipped with the same mechanical fastening elements used in diapers. Instead, the respective waist regions already are attached to one another by any of a variety of mechanisms, and the garment typically is simply pulled on by the wearer. The prior fastening mechanisms that enable one to roll up the used article, fasten it to secure its configuration, and then dispose of the diaper, therefore are not suitable for use in disposable pants-type diapers.

Disposable pants-type diapers have employed in the past various tape arrangements to secure a rolled up garment, and thereby enable easy disposal. U.S. Pat. No. 5,626,573, the disclosure of which is incorporated by reference herein in its entirety, discloses a vertically extending fastening tape having an adhesive section provisionally bonded to the outer surface of the garment. The upper end or lower end of the tape is permanently bonded to the outer surface of the garment. U.S. Pat. No. 5,807,371, the disclosure of which is incorporated by reference herein in its entirety, discloses an extensible fastening tape. The tape can be stretched after peeling a portion of the tape from the base, and then extending it around the garment to secure it in a rolled up configuration for easy disposal.

U.S. Pat. No. 6,063,066, the disclosure of which is incorporated by reference herein in its entirety, discloses a tape fastener that includes an extendible intermediate section and a pair of inelastic and adhesive distal sections. The tape can be extended and wrapped wholly or partially around the rolled up garment and then secured to the outer cover of the garment by the inelastic and adhesive distal end sections. U.S. Pat. No. 6,210,386, the disclosure of which is incorporated by reference herein in its entirety, discloses an auxiliary flap instead of a tape. The auxiliary flap is elastic circumferentially and has it circumferentially opposite side edges unitized with the waist region along transversely opposite side edges of this waist region to allow the garment to be rolled up and then secured in its rolled up configuration.

While the tape fasteners disclosed in the aforementioned documents may be useful in securing the garment in a rolled up configuration, that is the extent of their utility. They often detract from the physical appearance of the garment, and sometimes interfere with the elasticity of various portions of the garment, if the tape is placed at or near the waist opening or one of the leg openings.

The description herein of various advantages and disadvantages associated with known apparatus, products, and methods is in no way intended to limit the scope of the present invention. Indeed, some aspects of the present invention may contain similar apparatus, products, or methods as those that are known, yet not suffer from the aforementioned disadvantages.

### SUMMARY OF THE INVENTION

It would be desirable to provide an absorbent garment having a disposal securing system attached to an outer cover thereof that does not distract from the aesthetics of the garment. It also would be desirable to provide an absorbent garment having a disposal securing system that provides relevant information to the caregiver or wearer. Various features of embodiments of the present invention satisfy these needs.

These and other features of the invention can be achieved by an absorbent garment including a top sheet, a back sheet and an absorbent core at least partially disposed between the top sheet and the back sheet. The top sheet, back sheet and absorbent core define a first waist region, a second waist, and a crotch region there between. The garment also has a longitudinal centerline extending from the first waist region, through the crotch region and to the second waist region, and a lateral centerline orthogonal to the longitudinal centerline. The disposable absorbent garment also has disposed on an outer surface of the back sheet a disposal fastening mechanism that is capable of maintaining the garment in a rolled up configuration after the garment is rolled up. The disposal fastening mechanism includes visible indicia disposed thereon. It is preferred that the visible indicia provide information to the caregiver or wearer of the disposable absorbent garment, or provide a graphic, or provide indicia indicating the origin of the article.

In accordance with another feature of an embodiment of the invention, there is provided a method of making an absorbent garment that includes providing a top sheet material, a back sheet material, and an absorbent core to a garment forming station. The method also includes disposing the absorbent core at least partially between the top sheet material and the back sheet material at the garment forming station. The method further includes providing a disposal fastening mechanism having visual indicia disposed thereon, and disposing the disposal fastening mechanism on an outer surface of the back sheet either upstream or downstream from the garment forming station. The method preferably includes disposing the disposal fastening mechanism in registration on an outer surface of the back sheet material such that the visible indicia disposed thereon will be recognizable by a caregiver, purchaser, or wearer.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a front plan view of a pants-type absorbent garment with portions of the back sheet removed to reveal inner components of the garment;

FIG. 2 is a schematic of the various parts of a pants-type absorbent garment;

FIG. 3 is a back plan view of a pants-type absorbent garment showing a disposal fastening mechanism of the invention;

FIG. 4 is an illustration of an apparatus and method useful for making an absorbent garment of the invention;

FIG. 5a includes top and side views of an embodiment of a disposal fastening mechanism when secured to the absorbent garment during use; FIG. 5b includes top and side views of the embodiments shown in

FIG. 5a after the disposal fastening mechanism has been employed to secure the garment in its rolled up configuration; and

FIG. 6 is an illustration of an absorbent garment in a rolled up configuration that has been secured by a disposal fastening mechanism of the invention.

### DETAILED DESCRIPTION OF THE PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

Absorbent garments and diapers may have a number of different constructions. In each of these constructions it is generally the case that an absorbent core is at least partially disposed between a liquid pervious, body-facing top sheet, and a liquid impervious, exterior back sheet. In some cases, one or both of the top sheet and back sheet may be shaped to form a pants-like garment. In other cases, the top sheet, back sheet and absorbent core may be formed as a discrete assembly that is placed on a main chassis layer and the chassis layer is shaped to form a pants-like garment. The garment may be provided to the consumer in the fully assembled pants-like shape, or may be partially pants-like and require the consumer to take the final steps necessary to form the final pant-like shape. In the case of training pants-type garments and most adult incontinent products, the garment is provided fully formed with factory-made side seams and the garment is donned by pulling it up the wearer's legs. In the case of diapers, a caregiver usually wraps the diaper around the wearer's waist and joins the side seams manually by attaching one or more adhesive or mechanical tabs, thereby forming a pant-like structure.

For clarity, the present invention is described herein only with reference to a pants-type garment in which the top sheet, back sheet and absorbent core are assembled onto a chassis layer that forms a pants-like garment, although the invention may be used with other constructions, and it is readily apparent and understood that this is not intended to limit the invention. The present invention may be used with any other absorbent garment that can be rolled up and disposed of by a disposal fastening mechanism as described herein.

The term “component” can refer, but is not limited to designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent composite core. It is understood that additional layers may be present between the absorbent composite core and the top sheet and back sheet, and that additional layers and other materials may be present on the side opposite the absorbent composite core from either the top sheet or the back sheet.

The expression “visible indicia” denotes any type of character, component, graphic, or the like that is visible to a purchaser, wearer, caregiver, etc., when the pants-type absorbent garment is retrieved from its packaging, and/or when the garment is placed on a wearer. The visible indicia most likely is not visible to the ordinary wearer, however, when the garment is being worn since it preferably is disposed on the outer cover of the garment in the back region. The indicia is visible by virtue of it being printed on the outer surface of the disposal fastening mechanism, or on an inner layer that is covered with a relatively transparent or translucent outer layer.

Any indicia can be used in the invention including any type of character, component or graphic. For example, the indicia may simply denote the front or back of the garment which will indicate to the person donning the garment which portion is in the front or back. The indicia also may include letters or characters indicating the origin of the garment, such as a tradename, trademark, etc. The indicia also may be a graphical representation of a character or an object, including the characters and objects disclosed, for example, in U.S. Pat. Nos. 6,307,119 and 6,297,424, the disclosures of which are incorporated by reference herein in their entirety.

The present invention relates generally to absorbent garments, and in particular to a pants-type absorbent garment that contains a top sheet, a back sheet, and an absorbent core at least partially disposed between the top sheet and the backsheet. The absorbent garment of the invention preferably has a front waist region, a rear waist region and a crotch region positioned between the front and rear waist regions. Those skilled in the art recognize that “front” and “rear” in the context of the invention denote for clarity purposes only the front and rear of a user, and that the absorbent garment could be reversed whereby the previously described “front” portion becomes the rear portion, and vice versa.

The garment also has a longitudinal centerline extending from the first waist region, through the crotch region and to the second waist region, and a lateral centerline orthogonal to the longitudinal centerline. The disposable pants-type absorbent article also has disposed on an outer surface of the back sheet a disposal fastening mechanism that is capable of maintaining the garment in a rolled up configuration after the garment is rolled up. The disposal fastening mechanism includes visible indicia disposed thereon. It is preferred that the visible indicia provide information to the caregiver or wearer of the disposable pants-type absorbent garment, or provide a graphic, or provide indicia indicating the origin of the article.

The invention also encompasses a method of making a disposable pants-type absorbent garment that includes providing a top sheet material, a back sheet material, and an absorbent core to a garment forming station. The method also includes disposing the absorbent core at least partially between the top sheet material and the back sheet material at the garment forming station. The method further includes providing a disposal fastening mechanism having visual indicia disposed thereon, and disposing the disposal fastening mechanism on an outer surface of the back sheet either upstream or downstream from the garment forming station. The method preferably includes disposing the disposal fastening mechanism in registration on an outer surface of the back sheet material such that the visible indicia disposed thereon will be recognizable by a caregiver, purchaser, or wearer.

In the disposable pants-type absorbent garment of the invention, leg elastics preferably are provided along the leg openings for securely holding the leg openings against the thighs of the wearer to improve containment and fit. A pair of stand-up leg gathers or waist containment flaps may be attached to or formed from the body's side surface of the top sheet. Other elastic elements may be disposed in or on the absorbent garment to provide a firmer fit around the tummy and/or waist of the wearer.

The invention now will be described with reference to the attached drawings illustrating preferred embodiments of the invention. For clarity, features that appear in more than one Figure have the same reference number in each Figure.

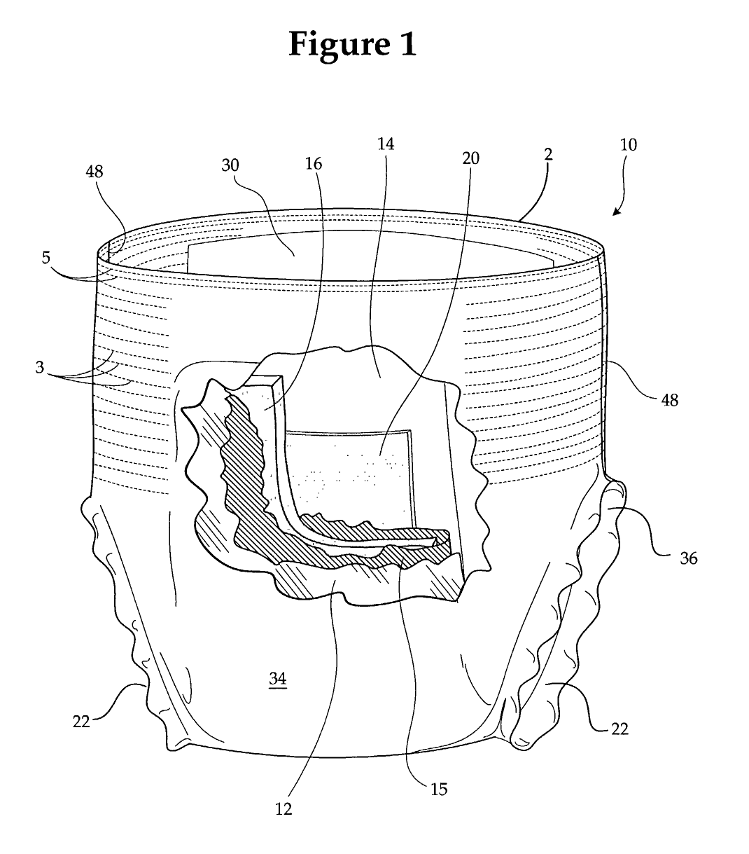

FIG. 1 depicts an embodiment of the present invention as it appears when worn by a user, with the main body partially cut away to show the absorbent core 16, and other internal components including, for example, an additional layer 20 (e.g., fluid transfer or handling layer, fluid acquisition layer, additional storage layer, wicking layer, and the like), and a tissue layer 15 surrounding the core 16. In the depicted embodiment, the garment 10 is comprised of a main body 34 having an exterior facing moisture impervious outer layer 12 or “back sheet,” and a moisture pervious body-contacting inner layer 14 or “top sheet.” An absorbent core 16 is at least partially disposed between the top sheet 14 and the back sheet 12. In the embodiment depicted in FIG. 1 the back sheet 12, top sheet 14, and core 16 comprise the main body 34 of the garment, however in another embodiment of the invention the main body may be made from a separate sheet and the back sheet 12, top sheet 14, and core 16 may be assembled separately then attached to the main body.

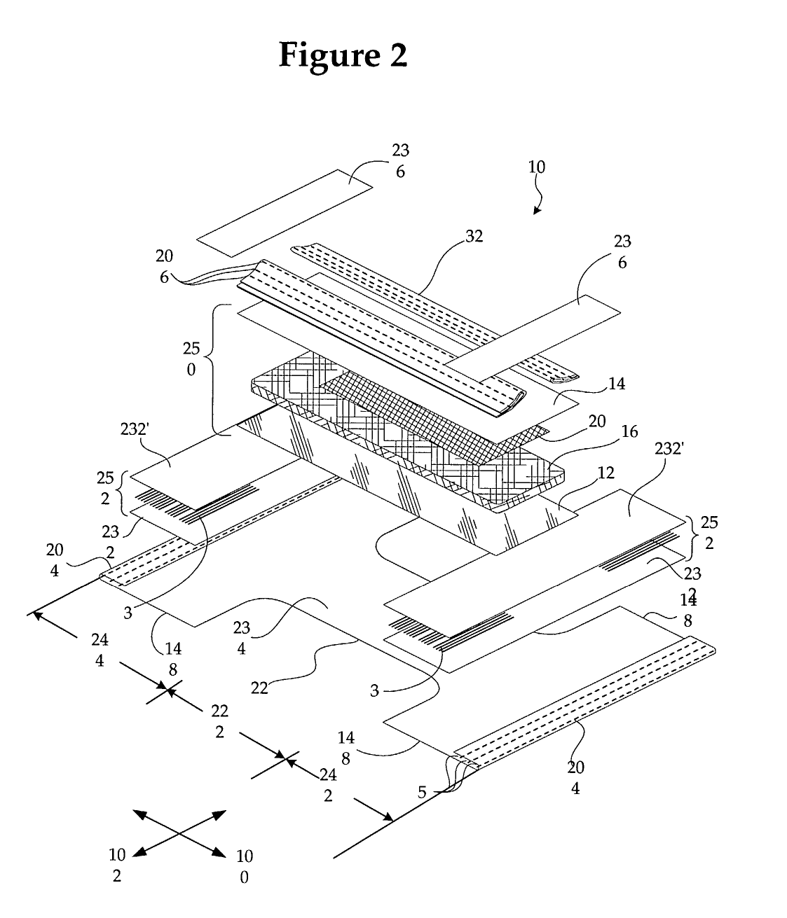

In the embodiment of the present invention depicted in FIG. 1, the garment 10 further comprises various mechanisms for improving the fit of the garment 10 such as leg gathers 36 and standing leg gathers 32 (see, FIG. 2). Such gathers can be used to contract the leg holes 22 around the wearer's legs and body to prevent leakage. A garment 10 of the present invention may also comprise elastic or other fitting devices in the waist portions or other portions of the main body to help contain body exudates.

The various parts of the garment 10 are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, or any other method of affixation known or hereafter discovered. All of the parts may be joined to each adjacent part, but some parts may not be joined to others. In one embodiment, the top sheet 14 and back sheet 12 are bonded to one another around their perimeter regions, thereby at least partially encasing and holding the absorbent core 16 in place without having to directly join the absorbent core 16 to any parts of the garment 10. The top sheet 14 or back sheet 12 also may be operatively associated with the absorbent core 16. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining means that maintains the structural integrity of the garment 10 for the duration of its use.

FIG. 2 is an exploded view of an embodiment of the present invention with elastic members shown in the elongated position for clarity, and the garment laid flat. The garment 10 has a longitudinal axis 100 corresponding approximately to the rear-to-front axis of the wearer, and a lateral axis 102, orthogonal to the longitudinal axis 100, and corresponding approximately to the side-to-side axis of the wearer. In one embodiment of the invention the lateral axis 102 of the garment 10 is approximately parallel with the machine direction of the garment 10.

In the embodiment of the invention depicted in FIG. 2, the main body 250 of the garment comprises a back sheet 12 and top sheet 14 having substantially identical dimensions. Those skilled in the art will recognize, however, that back sheet 12 and top sheet 14 need not have substantially identical dimensions; rather, either material may be smaller or larger than the other. The main body 250 of the absorbent garment 10 preferably is covered by an outer cover, more preferably, a chassis layer or layers 234. The perimeter of the chassis layer or layers is defined by laterally extending front and rear waist edges 204, longitudinally extending left and right front side edges 148 and left and right rear side edges 148, and leg holes 22. The laterally extending front waist edge 204, left and right front side edges 148, and at least part of leg holes 22 form a front waist region 242. The laterally extending rear waist edge 204, left and right rear side edges 148, and at least part of leg holes 22 form a rear waist region 244. The remaining portions of leg holes 22 form the crotch region 222.

Throughout this description, the terms “front,” “rear,” “left,” and “right” merely denote location relationships for purposes of explanation and clarity, and they generally relate to the location depicted in the drawings. Those skilled in the art appreciate that the front and rear of the absorbent garment may be reversed, as well as the left and right sides of the absorbent garment 10 depending upon the vantage point of the viewer.

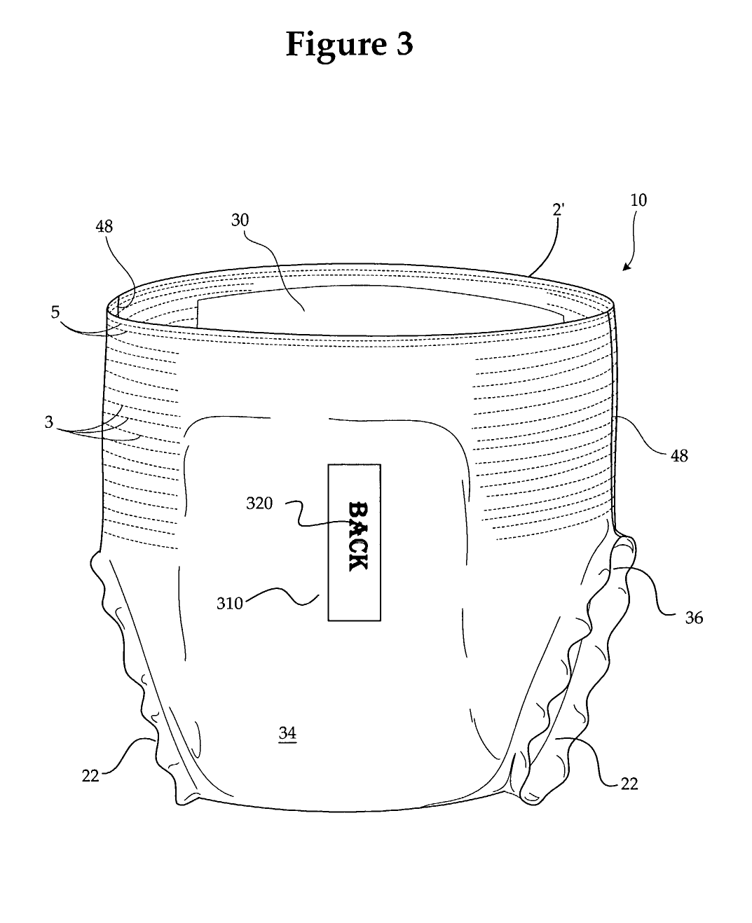

To form the absorbent garment shown in FIG. 2 into a pants-type absorbent garment of the type shown in FIGS. 1 and 3, the lateral edge portions 148 may be joined during or after manufacture by any mechanism known in the art or by a combination of such mechanisms.

Examples of such mechanisms include: applying adhesives such as hot melt adhesives and construction adhesives, chemical or solvent bonding, stitching, heat bonding, autogenous bonding, and, preferably, ultrasonic welding. The lateral edge portions 148 also may be held proximal to one another or in an overlapping relationship during use by a fastener, such as a hook-and-loop fastener or adhesive fastener, as are well known in the art. When the lateral edge portions 148 are joined, leg hole cutouts along the lateral edges of the garment 10 form leg holes 22, and the longitudinal ends 104 of the garment 110 form a waist encircling edge 2 (FIG. 1).

The top sheet 14 and back sheet 12 may be constructed from a wide variety of materials known in the art. The invention is not intended to be limited to any specific materials for these components. The top sheet 14 and back sheet 12 can be shaped and sized according to the requirements of each of the various types of absorbent garment, or to accommodate various user sizes. In an embodiment of the invention in which the garment 10 is a diaper or an adult incontinence brief, the combination of top sheet 14 and back sheet 12, may have an hourglass shape, as seen in FIG. 1, or may have a rectangular, trapezoidal, “T” shape, or other shape.

Due to the wide variety of backing and liner sheet construction and materials currently available, the invention is not intended to be limited to any specific materials or constructions of these components. The back sheet 12 preferably is made from any suitable pliable liquid-impervious material known in the art. The selection and manufacture of such materials is well known in the art, and is disclosed, for example, in U.S. Pat. No. 6,123,694 issued to Peniak et al., and U.S. Pat. No. 6,176,952 issued to Maugans et al., the disclosure of each of which is incorporated herein by reference in its entirety. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 12 may be pigmented with, for example, titanium dioxide, to provide the garment 10 with a pleasing color or to render the back sheet 12 opaque enough that exudates being contained by the garment 10 are not visible from outside the garment. In addition, the back sheet 12 may be formed in such a manner that it is opaque, for example, by using various inert components in the polymeric film and then biaxially stretching the film. Other back sheet materials will be readily apparent to those skilled in the art. The back sheet 12 preferably has sufficient liquid imperviousness to prevent any leakage of fluids. The required level of liquid imperviousness may vary between different locations on the garment 10.

The back sheet 12 may further comprise separate regions having different properties. In a preferred embodiment, portions of the back sheet 12 are air-permeable to improve the breathability, and therefore comfort, of the garment 10. The different regions may be formed by making the back sheet 12 a composite of different sheet materials, chemical treatment, heat treatment, or other processes or methods known in the art. Some regions of the back sheet 12 may be fluid pervious. In one embodiment of the invention, the back sheet 12 is fluid impervious in the crotch region 222, but is fluid pervious in portions of the first and second waist regions 242, 244. The back sheet 12 may also be made from a laminate of overlaid sheets of material.

The moisture-pervious top sheet 14 can be comprised of any suitable relatively liquid-pervious material known in the art that permits passage of liquid there through. Non-woven liner sheet materials are exemplary because such materials readily allow the passage of liquids to the underlying absorbent core 16. Examples of suitable liner sheet materials include non-woven spun bond or carded webs of polypropylene, polyethylene, nylon, polyester and blends of these materials.

The back sheet 12 may be covered with a fibrous, non-woven fabric or chassis layer(s) 234 such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The chassis layer 234 also may comprise a non-woven polyethylene or polypropylene sheet, a polyethylene film, or any other suitable garment material known in the art or hereafter discovered. All or part of the chassis layer 234 may comprise a liquid pervious or liquid impervious material or a may be zone-treated to be partially liquid pervious or impervious. The chassis layer 234 may be stretched in one or more directions during the manufacturing process, thereby reducing its elasticity in the direction of stretch.

Alternatively, the back sheet 12 may comprise three panels wherein a central poly back sheet panel is positioned closest to absorbent core 16 while outboard non-woven breathable side back sheet panels are attached to the side edges of the central poly back sheet panel. Alternatively, the back sheet 12 may be formed from microporous poly coverstock for added breathability.

Although not illustrated in the drawings, the top sheet 14 may be formed of three separate portions or panels. Such an embodiment is disclosed, for example, in U.S. Pat. No. 5,275,590 issued to Huffman et al., which is incorporated herein by reference in its entirety, and in a manner consistent with the present invention. Those skilled in the art will recognize, however, that top sheet 14 need not be made of three separate panels, and that it may be comprised of one unitary item, or of a top sheet material 14, with separate standing leg gathers material 32 attached thereto, as shown in FIG. 2. A first top sheet panel may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet panel preferably extends from substantially the second waist region 244 to the first waist region 242, or a portion thereof.

The second and third top sheet panels (e.g., outer top sheet panels), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 12. The material for the outer top sheet portions or panels preferably is polypropylene and can be woven, non-woven, spun bonded, carded or the like, depending on the application.

The respective top sheet panels may be attached to one another by, e.g., an adhesive. At the point of connection with the outer edges of the central top sheet panel and the inner edges of the outer top sheet panels extend upwardly to form waste containment flaps 32 (waist containment flaps 32, or standing leg gathers 32, are shown as separate elements in FIG. 2, but they need not be). The waste containment flaps 32 preferably are formed of the same material as the outer top sheet panels, as in the embodiment shown. They most preferably are an extension of the outer top sheet panels.

The waste containment flaps 32 may be treated with a suitable surfactant to modify their hydrophobicity/hydrophilicity as desired, and they may be treated with skin wellness ingredients to reduce skin irritation. Alternatively, the waste containment flaps 32 may be formed as separate elements and then attached to the body side liner or top sheet 14. In this alternative embodiment, the central top sheet panel may extend past the connection point with the waste containment flaps 34, and even extend to the periphery of the back sheet 12.

The waste containment flaps 32 preferably include a portion that folds over onto itself to form a small enclosure. At least one, and depending on the size of the enclosure sometimes more than one, elastic element 206 may be secured in the enclosure in a stretched condition. As is well known in the art, when the flap elastic elements 206 attempt to assume the relaxed, unstretched condition, the waste containment flaps 32 rise above the surface of the center of the top sheet 14, as shown in FIG. 2.

The top sheet 14 may be made of any suitable relatively liquid-pervious material currently known in the art or later discovered that permits passage of a liquid there through. Examples of suitable top sheet materials include non-woven spun-bonded or carded webs of polypropylene, polyethylene, nylon, polyester and blends of these materials, perforated, apertured, or reticulated films, and the like. Non-woven materials are exemplary because such materials readily allow the passage of liquids to the underlying absorbent core 16. The top sheet 14 preferably comprises a single-ply non-woven material that may be made of carded fibers, either adhesively or thermally bonded, spun bonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 14, and other top sheet materials will be readily apparent to those skilled in the art.

The top sheet 14 may further comprise several regions having different properties. In one embodiment of the present invention, the laterally distal portions of the top sheet 14, especially those used to make second and third top sheet panels, preferably are substantially fluid impervious and hydrophobic, while the remainder of the top sheet 14 (e.g., central top sheet panel) is hydrophilic and fluid pervious. Different top sheet properties, such as fluid perviousness and hydrophobicity, may be imparted upon the top sheet 14 by treating the top sheet 14 with adhesives, surfactants, or other chemicals, using a composite of different materials, or by other means. The top sheet 14 may also be made from a laminate of overlaid sheets of material. The top sheet 14 also may be treated in specific areas like the crotch region, with skin wellness ingredients such as aloe, vitamin E, and the like.

As noted elsewhere herein, the top sheet 14 and back sheet 12 may be substantially coterminous, or they may have different shapes and sizes. The particular design of the top sheet 14 and back sheet 12 may be dictated by manufacturing considerations, cost considerations, and performance considerations. Preferably, the top sheet 14 is large enough to completely cover the absorbent core 16, and the back sheet 12 is large enough to prevent leakage from the garment 10. The design of top sheet 14 and back sheet 12 is known in the art, and a skilled artisan will be able to produce an appropriate top sheet 14 and an appropriate back sheet 12 without undue experimentation.

The top sheet 14 and the back sheet 12 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as Cycloflex as sold by National Starch, a corporation headquartered in Bridgewater, N.J., is used to join the top sheet 14 to the back sheet 12. The particular joining method may be dictated by the types of materials selected for the top sheet 14 and back sheet 12.

In one embodiment of the present invention, the top sheet 14 is operatively associated with the back sheet 12 around the perimeter of the top sheet 14. In this embodiment, the top sheet 14 and back sheet 12 may be operatively associated with one another by using hot melt adhesives, ultrasonic bonding, or any other suitable method known in the art. Also in this embodiment, the top sheet 14 and back sheet 12 may be bonded to one another in substantially all areas not having intermediately placed parts, such that some or all of the intermediately placed, or “sandwiched,” parts are physically captured between the top sheet 14 and back sheet 12, but not bonded to the back sheet 12 or top sheet 14.

An absorbent core 16 preferably is disposed between the inner surfaces of the back sheet 12 and the top sheet 14. The absorbent core 16 may be comprised of one or more layers of material, such as an absorbent layer for storing fluids and an acquisition layer for distributing fluids. Such multiple layer absorbent cores are known in the art and disclosed in U.S. Pat. No. 5,439,458 issued to Noel et al., which is incorporated herein by reference in its entirety, and in a manner consistent with the present invention.

The absorbent core 16 may be made from any absorbent material or materials known in the art. In one embodiment of the invention, the absorbent core 16 comprises wood fibers or other fibers such as tow fibers, chemical wood pulp, or any other suitable liquid absorbing material, such as commercially available fluff pulp or fluffed bleached kraft softwood pulp. In another embodiment of the invention, the absorbent core 16 comprises a combination of a porous fibrous web and super absorbent particles. Such absorbent cores are known in the art and are disclosed, for example, in U.S. Pat. No. 5,281,207 issued to Chmielewski et al., which is incorporated herein by reference in its entirety. In such an embodiment, the absorbent core 16 may be surrounded by a liquid pervious tissue over-wrap 15 (FIG. 1), or other material.

The absorbent core 16 also may be a laminate material, as described in U.S. Pat. No. 6,068,620, the disclosure of which is incorporated by reference herein in its entirety. Such laminate materials typically include outer tissue layers 15 (FIG. 1) surrounding a central fibrous layer 16 that contains a high percentage by weight of superabsorbent polymer (SAP). Any type of SAP can be used in this embodiment, or in any absorbent core 16 that is useful in this invention. The SAP generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Any SAP now known or later discovered can be used in the absorbent core 16 of the invention. Commercially available SAPs include a starch modified superabsorbent polymer available under the trade name SANWET® from Hoechst Celanese Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; FAVOR manufactured by Stockhausen Inc.; HYSORB from BASF Atkienqesellshaft, Ludwigshafen, Germany; AQUA KEEP SA60S, manufactured by Seitetsu Kagaku Co., Ltd.; DIAWET, commercially available from Mitsubishi Chemicals, Japan; FLOSORB, available from SNF Floerger, France, AQUALIC, available from Nippon Shokubai, Osaka, Japan.

The SAP may be provided in any particle size, and suitable particle sizes vary greatly depending on the ultimate properties desired. For example, a fine particulate rather than a coarse particulate may be used in the invention, and preferably a fine particulate that passes through an about 200 mesh screen may be used.

The absorbent core 16 may be surrounded by a liquid pervious tissue over-wrap 15 (FIG. 1), or other material, which may be treated to be hydrophobic or hydrophilic, or to have other properties. The absorbent core 16, and any tissue wrap enclosing it, may be folded, crimped, thermally bonded, or otherwise manipulated to provide additional benefits. It is envisioned that a variety of folding patterns may be employed to provide additional fluid handling capabilities. For example, the absorbent core 16 may be folded into a U shape, a C shape, a G shape, a Z shape, or other shapes, as viewed along the longitudinal axis 100, to provide fluid handling channels, multiple layers of absorbent material, or other benefits. Folded absorbent cores are discussed, for example, in U.S. Pat. No. 6,068,620.

The absorbent core 16 generally is elongated along the longitudinal axis 100 of the garment, and may extend along either or both of the lateral axis 102 and the longitudinal axis 100 to the outer perimeter of the garment. In the embodiment depicted in FIGS. 1 and 2, the absorbent core 16 is substantially rectangular in shape, however, it also may have rounded ends or other shapes, such as an “I” shape or a “T” shape. The absorbent core 16 also may have channels, grooves or pockets, and may have a varying thickness. In an embodiment having a channeled or pocketed absorbent core 16, such channels or pockets may be substantially vacant, or may be filled with additional SAP or additional supplemental absorbent cores having similar or different properties than the absorbent core 16.

As mentioned previously, the various parts of the garment 10 preferably are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, autogenous bonding, or any other method of affixation known or hereafter discovered. U.S. Pat. No. 4,919,738 issued to Ball et. al. discloses a method of autogenous bonding, and its disclosure is herein incorporated by reference in its entirety in a manner consistent with the invention. All of the parts may be joined to each adjacent part, but some parts may not be joined to others.

The core assembly 250 may comprise additional layers 20 of material that may reduce rewet of the top sheet 14, reduce strikethrough times or otherwise improve the absorbency, dryness and other properties of the garment 10. Examples of the one or more additional layers 20 include any layer selected from a fluid acquisition layer, a distribution layer, an additional fibrous layer optionally containing SAP, a wicking layer, a storage layer, a dryness layer, a softness layer, or combinations and fragments of these layers. Such layers may be provided to assist with transferring fluids to the absorbent core 16, handling fluid surges, preventing rewet, containing absorbent material, improving core stability, or for other purposes. Skilled artisans are familiar with the various additional layers 20 that may be included in an absorbent article, and the present invention is not intended on being limited to any particular type of materials used for those layers. Rather, the invention encompasses all types of wicking layers, all types of distribution layers, etc., to the extent that type of layer is utilized.

The core assembly 250 may be attached to the chassis layer 234 by any mechanism known in the art, such as by ultrasonic bonding or by the use of lines of hot melt adhesive. The bond between the core assembly 250 and the chassis layer 234 may be reinforced by laterally-extending end strips 236 that preferably are applied over the longitudinal ends of the core assembly 250 and bonded to the underlying structure of the garment 10. The end strips 236 also may hold the ends of the standing leg gathers 32 so that the standing leg gathers 32 face inward or outward. Such end strips 236 preferably comprise a fluid pervious non-woven material, but may be fluid impervious or a material other than a non-woven material. Such materials are known in the art. The end strips 236 also may help prevent the longitudinal flow of exudates past the ends of the core assembly 250, particularly if the edges of the non-woven strips overlying the core assembly 250 are left un-bonded so that they form pockets to hold exudates.

Waist elastics 5 and tummy elastics 3 may be incorporated into the garment 10 to contract the garment 10 about the wearer's abdomen. Such elastics are typically stretched as they are joined to the garment 10 so that the contraction of the elastics causes the garment 10 to contract about the wearer. The elastics also may be applied in an unstretched state and then mechanically stretched to create an elasticized region in the garment (often called a zero-strain laminate). The elastics also may be applied in an inelastic state and then heat activated to cause them to become elasticized. The elastics 3, 5, and 206 may be made from natural or synthetic rubber, elastomers, LYCRA® elastomer (available from E.I. DuPont de Nemours and Company, a business having offices in Wilmington, Del.), polyurethane, heat shrinkable polymer ribbons, or any other suitable elastic material or composite. Such materials are known in the art.

In a preferred embodiment, the waist elastics 5 are located proximal to one or both longitudinal ends 204 of the chassis layer 234, and are thereby located along the waist encircling edge of the fully assembled garment 10. In such an embodiment, the waist elastics 5 may be located on one side of the chassis layer 234, within a fold in the chassis layer 234 (as shown in FIG. 2), or otherwise fixed in the proximity of the longitudinal ends 204. U.S. Pat. No. 4,515,595 issued to Kievit et. al. and U.S. Pat. No. 4,816,025 issued to Foreman illustrate other embodiments of elasticized waist features of absorbent garments, and are hereby incorporated by reference in their entirety.

Tummy elastics 3 also may be disposed in the garment 10 between the longitudinal ends 204 and the leg opening cutouts 22 to thereby be positioned across the wearer's stomach. The tummy elastics 3 may be attached directly to the chassis layer 234 or may be disposed between a pair of carrier layers 232, 232′ to form tummy elastic assemblies 252 that are attached to the chassis layer 234. The tummy elastics 3 may be located on the interior or exterior side of the chassis layer 234, and may be covered by additional layers of material. In a preferred embodiment, the tummy elastics 3 are affixed between a pair of carrier layers 232, 232′. The carrier layers 232, 232′ preferably comprise non-woven materials, but may be made of any suitable material, and may be liquid pervious or liquid impervious. The carrier layers 232, 232′ are preferably gas pervious to allow the garment 10 to “breathe.”

In one embodiment, the tummy elastics 3 may extend across the entire width of the garment 10. In a preferred embodiment, shown in FIGS. 1 and 2, the tummy elastics 3 extend across the lateral sides of the garment 10, but not across the portion of the garment 10 overlying the absorbent core 16. Such a preferred embodiment may provide improved fit and comfort and improve the garment's appearance. U.S. Pat. No. 5,449,353 issued to Watanabe et. al. and U.S. Pat. No. 5,749,865 issued to Yamamoto et al. illustrate other embodiments of elasticized waist features of absorbent garments, and are incorporated herein by reference in their entirety.

The elastics 3, 5, 206 or any other elastics may be joined to the garment 10 by the use of a flexible adhesive or other suitable joining method. Suitable adhesives include HL-1258 by H. B. Fuller Company of St. Paul, Minn.; Findley 2031 and H2587-01 by Ato Findley Inc. of Wauwatosa, Wis.; and NS34-5665 by National Starch Co of Bridgewater, N.J. Adhesives that may be used to secure elastic elements to the absorbent garment include 34-578A by National Starch Co. of Bridgewater, N.J. In a preferred embodiment of the invention, the adhesive utilized includes HL 1486UZP, which is available from H. B. Fuller Company of St. Paul, Minn. This and other methods for attaching elastics to absorbent garments are known in the art.

As noted previously, it often is desirable for an absorbent garment to contract around various parts of the wearer's body to provide improved comfort and exudate containment. In a preferred embodiment of the present invention depicted in FIG. 2, the garment 10 further comprises at least one standing leg gather 32, and preferably at least 2 standing leg gathers 32, for improving the ability of the garment 10 to contain body exudates. The standing leg gathers 32 may be formed by incorporating a plurality of gather elastics 206 into folds in the top sheet 14 (not shown), or preferably may be provided as separate standing leg gather assemblies that are attached to the garment 10 near the leg hole cutouts 22 as shown in FIG. 2. The gather elastics 206 cause the standing leg gathers 32 to rise above the interior surface of the garment 10, thereby forming vertical curtains of material that help contain exudates. The standing leg gathers 32 may be liquid pervious or liquid impervious, and more than one pair of opposing standing leg gathers 32 may be provided.

Additional elastics (not shown) also may be incorporated into the chassis layer 234, top sheet 14 or back sheet 12 adjacent the leg hole cutouts 22 to form non-standing leg gathers 36 (FIG. 1), as is known in the art. Non-standing leg gathers 36 contract the garment 10 around the wearer's legs and body to prevent leakage. U.S. Pat. Nos. 3,860,003 and 4,081,301 issued to Buell, U.S. Pat. No. 4,695,278 issued to Lawson, U.S. Pat. No. 4,808,177 issued to Des Marais, U.S. Pat. No. 4,795,454 issued to Dragoo, and U.S. Pat. No. 4,938,755 issued to Foreman illustrate other embodiments of leg cuffs and gathers in absorbent garments, and the disclosures of these patents are hereby incorporated by reference in their entirety.

The absorbent garment 10 of the invention also preferably includes a fastening mechanism by which the front waist region 242 is associated with rear waist region 244 to form a waist hole 30. The fastening mechanism may comprise a permanent seal whereby the respective side edges 148 are attached to one another to form side seals 48 (FIG. 1). The absorbent garment 10 then can be pulled on and off like an undergarment or pair of pants. Any mechanism can be used to form the permanent seal 48. The use of the expression “permanent seal” in this context is not meant to encompass seals that cannot be broken, but rather permanent is meant to encompass sealing mechanisms that are not intended to be broken during normal use and application. The respective side edges 148 can be associated with one another to form seal 48 using techniques known in the art, including, for example, using adhesives such as hot melt adhesives and construction adhesives, chemical or solvent bonding, ultrasonic welding, stitching, heat bonding, or any other method of affixation known or hereafter discovered.

The absorbent garment 10 of the invention may also include a releasable fastening mechanism by which the front waist region 242 is associated with rear waist region 244 to form a waist hole 30. The releasable fastening mechanism may comprise a mechanism whereby the respective side edges 148 are releasably attached to one another to form side seals 48 (FIG. 1). Releasable fastening mechanisms are well known in the art, and may include tabs laterally extending from the laterally opposing rear side edges 148, adhesive strips, belts, and the like. The particular mechanism by which the respective parts of garment 10 may be adhered to one another include hook and loop type fasteners, pressure sensitive adhesives, snaps, clips, pins, and the like. Those skilled in the art are capable of manufacturing garment 10 to have either permanent or releasable fastening mechanisms, using the guidelines provided herein.

The absorbent garment 10 includes a disposal fastening mechanism. It is preferred in the present invention that the disposal fastening mechanism be disposed on the back or rear portion of the garment, as shown in FIG. 3. As shown therein, the disposal fastening mechanism 310 is disposed on the outer cover of the garment 10, say, on the outer surface of the back sheet material 12, or chassis layer 234. Disposal fastening mechanism 310 also could be placed near side seams 48, or in the front portion of garment 10. It is preferred in the invention that the disposal fastening mechanism 310 not be placed directly or indirectly on any substantial portion of elastic material 3, 5, etc. Skilled artisans will recognize various areas in which to place the disposal fastening mechanism 310 of the invention so that the mechanism can be used to secure the garment in a rolled up configuration.

Disposal fastening mechanism 310 includes visible indicia 320. Any indicia can be used so long as it is visible. The embodiment in FIG. 3 includes the letters “B-A-C-K” thereby indicating to the caregiver or user which side of the garment 10 is the back and which is the front. This facilitates easier donning of the garment 10. Other indicia 325 also could be used, such as a graphic or mark indicating the origin or manufacturer of the garment, (e.g., trademark), a colored graphic that is recognizable by children, (e.g., cartoon character, television character, card game characters, etc.), markings to indicate shading or seam lines to hide the disposal fastening mechanism 310, and the like. There are numerous indicia 320 that could be printed or otherwise disposed on the disposal fastening mechanism 310, and the invention is not intended to be limited to any particular visible indicia 320.

The disposal fastening mechanism 310 can be disposed on the garment 10 in any suitable manner. It is preferred that disposal fastening mechanism 310 be placed on garment 10 using cut-and-place techniques that are well known in the art, and then registering the disposal fastening mechanism 310 by placing it on a specific portion of garment 10. It is known to effect synchronous, in-line placement of absorbent core pads on a continuous web of material, as described in U.S. Pat. No. 5,415,716, the disclosure of which is incorporated by reference herein in its entirety. It further is known that graphics can be applied in registration on a moving sheet of material that ultimately is used in an absorbent garment by controlling the timing of placing the discrete graphic material. A number of documents describe placing graphic materials in registration on a moving web, including U.S. Pat. Nos. 6,165,306, 6,149,755, 6,095,218, 6,074,333, and 6,059,710, the disclosures of each of which are incorporated by reference herein in their entireties. Any of the methods and apparatus described in these documents can be used in the invention to place the disposal fastening mechanism 310 on the absorbent garment 10.

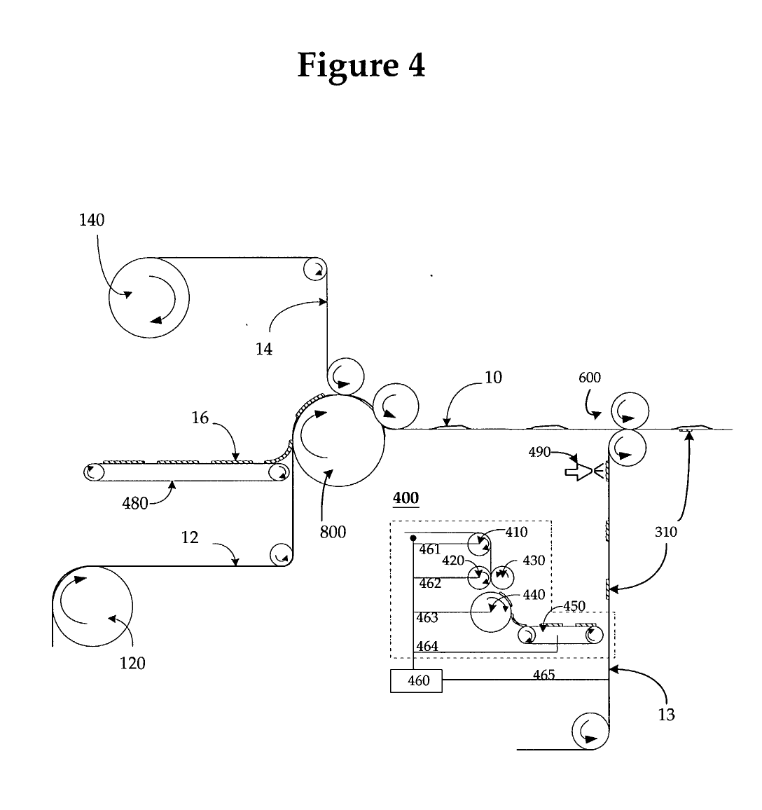

The methods and apparatus suitable for placing a disposal fastening mechanism 310 of the invention will be explained in further detail with reference to FIG. 4. Any cut-and-place apparatus 400 capable of cutting a material from a moving web at a first speed, and placing it on another moving web at a second speed different from the first speed can be used in the present invention. Suitable cut-and-place apparatus are disclosed in, inter alia, U.S. Pat. No. 6,165,306, 6,149,755, 6,095,218, 6,074,333, 6,059,710, and 5,415,716, the disclosures of each of which are incorporated by reference herein in their entireties. The simplified cut-and-place apparatus illustrated in FIG. 4 includes a feed roller system 410 that feeds a web of material containing the disposal fastening mechanism 310 at a first speed to the cut-and-place apparatus 400. Those skilled in the art will recognize that feed roller system 410 may be comprised of any number of component parts, such as a supply reel, a plurality of dancer rolls and edge guide rolls, and pull rolls to establish the first speed of the web material.

Feed roller system 410 feeds the web of material to a cutting station, illustrated in FIG. 4 as a pair of rollers in cutting engagement with one another. Any type of cutting device can be used in the present invention that is capable of controlled cutting of a moving web of material. FIG. 4 shows a rotatable knife roll 420 in cutting engagement with a rotatable anvil roll 430. The knife roll can be controlled to control cutting the web at the appropriate locations to form an appropriately sized additional layer 286.

Upon cutting, the disposal fastening mechanism is placed on a rotatable vacuum transfer drum 440 that alters the velocity of the moving web to the second speed so as to control its placement on, for example, an outer surface of garment 10, which is moving at a speed different from the speed at which the web containing the disposal fastening mechanism 310 is fed to cut-and-place apparatus 400. FIG. 4 illustrates an embodiment where the disposal fastening mechanism 310 first is transferred to moving web 13, and then placed in registration on garment 10 at placement forming station 600. Moving web 13 also may be chassis layer(s) 234, as will be appreciated by those skilled in the art. The rotatable vacuum transfer drum 440 can be any type of rotating drum capable of drawing a vacuum so that it can grab onto and hold the now severed disposal fastening mechanism 310, and ultimately transfer it to garment 10 via fastening mechanism conveyor 450, and moving web 13, for example. In this embodiment, fastening mechanism conveyor 450 transports the disposal fastening mechanism 310 at the second speed to the moving web 13, and preferably, the speed of the web that contains garment 10. Skilled artisans will appreciate, however, that rotatable vacuum transfer drum 440 could deposit the disposal fastening mechanism 310 on an outer surface of garment 10, without the use of the fastening mechanism conveyor 450, or moving web 13.

The cut-and-place apparatus 400 can be controlled by controller 460. Controller 460 monitors and controls the first speed at which the moving web is conveyed by monitoring and controlling feed roller system 410 via control 461, and by monitoring and controlling knife roll 420 and anvil roll 430 via control 462. Controller 460 also monitors and controls the second speed at which the severed disposal fastening mechanism 310 is conveyed by monitoring and controlling the rotatable vacuum transfer drum 440 via control 463, the fastening mechanism conveyor 450 via control 464, and by monitoring the speed of moving web 13 via control 465. Those skilled in the art will recognize that various modifications may be made to controller 460 to adequately control the first and second speeds such that the disposal fastening mechanism 310 is accurately placed on an outer surface of garment 10, and that any control system capable of such control can be used in the present invention. Using the guidelines provided herein, those skilled in the art are capable of designing a suitable controller 460 without undue experimentation.

FIG. 4 illustrates the disposal fastening mechanism 310 being disposed on an exterior surface of the back sheet 12, but the invention is in by no means limited to this particularly preferred embodiment and configuration. The disposal fastening mechanism could be disposed on the exterior surface of top sheet 14, or on an exterior surface of chassis layer(s) 234. The embodiment shown in FIG. 4 illustrates an adhesive applicator 490 applying adhesive to disposal fastening mechanism 310 to ensure its attachment to garment 10. It is preferred that the adhesive applicator 490 apply a permanent adhesive, as opposed to a peel-off adhesive to permanently adhere disposal fastening mechanism 310 to ensure its attachment to garment 10. Skilled artisan will recognize that permanent in the context described here simply denotes that the adhesive is applied in such a manner that disposal fastening mechanism 310 does not separate from garment 310 during normal use and disposal operations, as will be described in more detail below.

Any mechanism 490 capable of supplying an adhesive, albeit a spray adhesive, or one that is “rubbed” on, can be used in the invention. Suitable adhesives include any adhesive commonly employed in absorbent garments that is useful in adhering one or more components to together. It is particularly preferred to use construction adhesives, including HL-1258 by H. B. Fuller Company of St. Paul, Minn.; Findley 2031 and H2587-01 by Ato Findley Inc. of Wauwatosa, Wis.; and NS34-5665 by National Starch Co. of Bridgewater, N.J. Other adhesives that may be used in the invention include 34-578A, available from National Starch Co. of Bridgewater, N.J. Any of these adhesives may be used in all adhesive applications in the absorbent garment, or only in select applications as a construction adhesive for bonding parts of the garment as the top sheet, back sheet, absorbent core, and additional layer(s).

It is preferred in the invention that garment 10 be formed at garment forming station 800 prior to securing the disposal fastening mechanism 310 to garment 10. It will be appreciated, however, that disposal fastening mechanism could be disposed on an outer surface of back sheet 12, or top sheet 14 prior to garment forming station 800, using the techniques described herein.

The absorbent core 16 of the invention preferably is used immediately after it is formed as a component part of the absorbent garment 10. In this context, the absorbent cores 16 would be transported to garment forming station 800 via core conveyor 480 (or any other conveying device) where they will be disposed at least partially between a top sheet 14 and a back sheet 12. Top sheet material 14 may be supplied to forming station 800 by top sheet supply mechanism 140, which can be any supply mechanism capable of supplying top sheet 14 to garment forming station 800. Preferably, top sheet material 14 is supplied via a supply roller 140 and select feed or guide rollers. Back sheet material 12 likewise can be supplied to forming station 800 by back sheet supply mechanism 120, which can be any supply mechanism capable of supplying back sheet 12 to garment forming station 800. Preferably, back sheet material 12 is supplied via a supply roller 120 and select feed or guide rollers. Forming station 800 brings together the respective components of absorbent article 10 by disposing absorbent core 16 between top sheet material 14, and back sheet material 12. After placing disposal fastening mechanism on garment 10, the final absorbent garment 10 then may be cut and folded to the appropriate size and shape downstream from forming station 800.

The embodiment illustrated in FIG. 4 could include a number of additional devices used in preparing absorbent garments. For example, it is conventional to employ leg elastics 36, and standing leg gathers 32 (FIGS. 1-3). Leg elastics 36 could be supplied to garment forming station 800 by any suitable manner known in the art. It is conventional to apply adhesives to either the leg elastics 36 themselves, or to apply adhesives on back sheet 12 or top sheet 14, and then dispose the leg elastics 36 there between. The leg elastics 36 may be supplied as an elastic element, or as a laminate of elastic elements disposed between two outer layers, preferably outer non-woven materials.

Standing leg gathers 32 can be applied as a separate material to top sheet 14, and disposed on the exterior facing side (e.g., body facing side) of the top sheet 14. The standing leg gathers 32 can be applied either prior to, or downstream from, garment forming station 800 using techniques known in the art. The standing leg gathers 32 may be secured in place on garment 10 by placing outer sheets, preferably, outer non-woven sheets 236 over the longitudinal end portions of the standing leg gathers 32.

Other fastening elements also can be supplied and attached to garment 10 either prior to or downstream from garment forming station 800. It is preferred that fastening elements are secured to garment 10 after garment forming station 800. Additional layers 20 (FIG. 2) may be placed on or in garment 10 after forming station 800, or be disposed between top sheet 14 and back sheet 12 by supplying the additional layer(s) to garment forming station 800. These additional layer(s) 20 also may be cut-and-placed on top sheet 14 and/or back sheet 12 using a cut-and-place apparatus 400.

It also is typical in the industry to include a waist elastic system comprising one or more waist elastic materials 5. Waist elastics 5 preferably are supplied upstream of garment forming station 800, and thus disposed between the top sheet 14 and back sheet 12. Waist elastics 5 may, however, be supplied downstream from garment forming station 800, and placed within a fold at longitudinal ends 204 of an outer cover or chassis layer(s) 234 to be disposed outside the back sheet 12, using techniques known in the art. The waist elastic elements 5 may be supplied as a layer of material, or as elastic elements disposed between two outer materials. In a similar fashion, tummy elastics 3 may be supplied prior to, or downstream from garment forming station 800.

Disposal fastening mechanism 310 can be any mechanism that is capable of securing garment 10 in a rolled up configuration, preferably after use of the garment 10. For example, disposal fastening mechanism 310 can be a hook-and-loop type device, such as Velcro, or a web material with multiple perforations that allow one to tear the web and form a long string-like device to tie up the used garment. Alternatively, the disposal fastening mechanism 310 can be any of the adhesive tape devices or hook devices described in, inter alia, U.S. Pat. Nos. 4,963,140, 5,626,573, 5,807,371, 6,063,066, and 6,210,386, the disclosures of each of which are incorporated by reference herein in their entirety. It is most preferred that disposal fastening mechanism 310 be capable of partially peeling away from the outer cover of garment 10, extending longitudinally or laterally, depending on how the mechanism 310 is disposed on garment 10, and then adhering to a different portion of garment 10 after it has been rolled up. In this context, disposal fastening mechanism 310 may be comprised in part of an elastomeric material that can be stretched or extended, such as those described in U.S. Pat. Nos. 6,063,066 and 6,210,386.

The disposal fastening mechanism 310 preferably re-attaches to a portion of the rolled up garment 10 in a permanent manner. Permanent is used in this context in a manner similar to the context described previously. That is, after adhering disposal fastening mechanism 310 to the rolled up garment to maintain it in its rolled up configuration, the fastening mechanism should remain in place during normal disposal of the garment. If excessive force above and beyond that which would be encountered during normal disposal is applied, however, disposal fastening mechanism 310 may become partially or fully unattached.

There are any number of possible configurations for placing disposal fastening mechanism 310 on an outer surface of garment 10, such as placing it at various sites on the garment, in the front waist region 242, back waist region 244, near the waist 30 or leg hole 22, in the crotch region 222, longitudinally, laterally, or skewed at an angle. Disposal fastening mechanism 310 also can be designed to function in the manner described above to enable secure attachment after garment 10 is rolled up, in any of a variety of ways. Using the guidelines provided herein, those skilled in the art are capable of designing a suitable disposal fastening mechanism 310 and placing it on garment 10 in a suitable fashion.

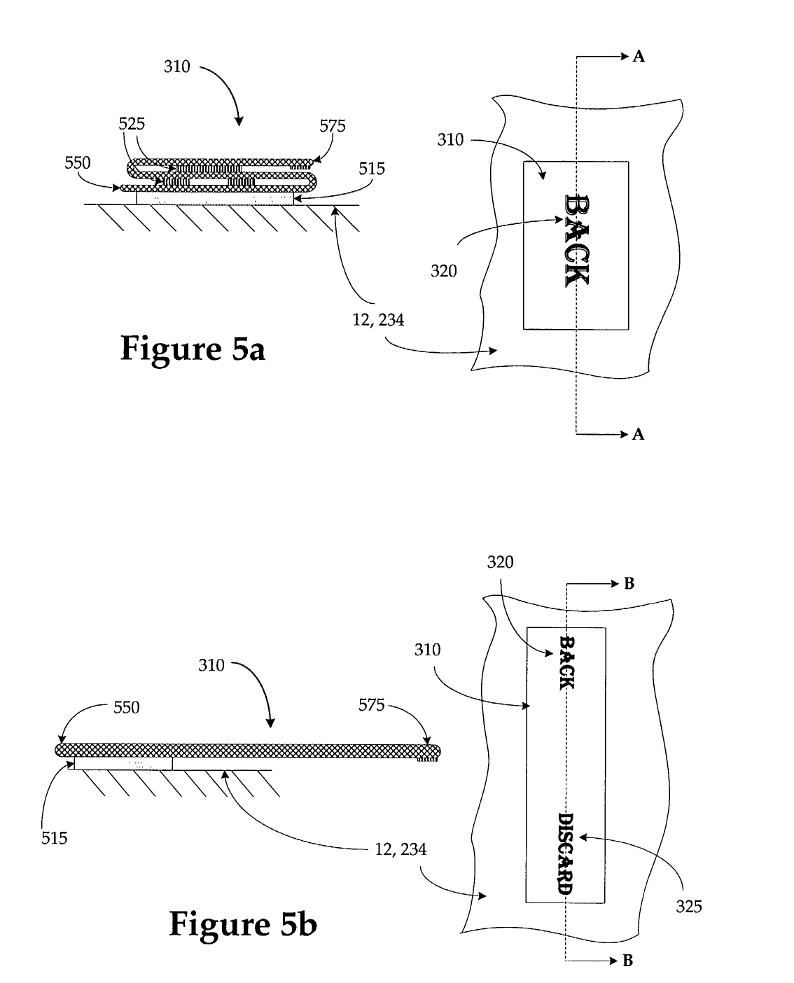

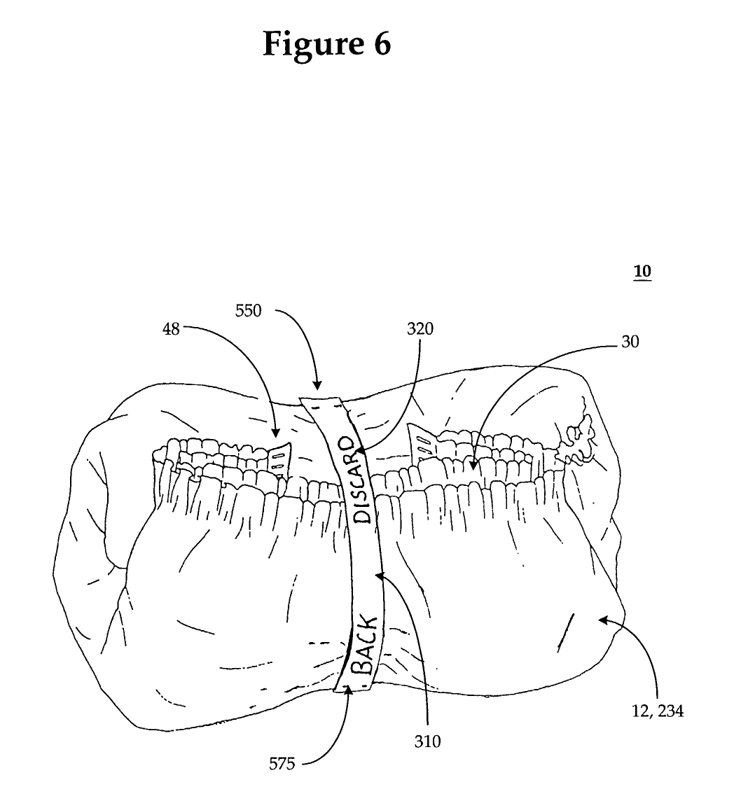

Preferred arrangements for disposal fastening mechanism 310 are illustrated in FIGS. 5a and 5b, and FIG. 6. The left-hand portion of FIG. 5a shows a side view of disposal fastening mechanism 310, as seen along line A-A from the top view shown at the right-hand portion of FIG. 5a. The left-hand portion of FIG. 5b shows a side view of disposal fastening mechanism 310, as seen along line B-B from the top view shown at the right-hand portion of FIG. 5b.

As shown in FIG. 5a, disposal fastening mechanism 310 can be placed on outer surface 12, 234 of garment 10 in a folded manner. In this embodiment, a first end 550 of disposal fastening mechanism 310 is permanently secured to the outer surface 12, 234, by means of a permanent seal 515. Any of the known adhesives described above that are capable of forming a permanent seal 515, as defined above, can be used for this purpose. Disposal fastening mechanism 310 then can be folded over onto itself any number of times and adhered with a peelable, or temporary adhesive 525. Again, any known adhesive capable of forming a temporary or peelable bond can be used in the invention. A second end 575 of disposal fastening mechanism 310 therefore is not permanently attached to outer surface 12, 234 of garment 10.

Garment 10 then can be rolled up starting with the crotch region 222, as shown in FIG. 6. The second end 575 of disposal fastening mechanism 310 can be peeled off from the outer surface 12, 234 of garment 10, and the peeling will stop by virtue of permanent seal 515. Second end 575 of disposal fastening mechanism 310 then can be stretched, if elastically extensible, or simply pulled to its desired length, wrapped tightly around the garment 10, preferably traversing both waist regions 242, 244, and waist hole 30, and secured to rolled up garment by second end 575. Those skilled in the art will recognize that the configurations illustrated in FIGS. 5a, 5b, and 6 merely represent one of any number of possible configurations for disposal fastening mechanism 310.

A feature of the present invention is that disposal fastening mechanism 310 include a visible indicia 320. Preferably, visible indicia 320 provides useful information or aesthetically pleasing graphical representations. One example of the type of indicia 320 that might be printed or otherwise disposed on disposal fastening mechanism 310 is shown in the figures. Here, the word “BACK” is imprinted on the disposal fastening mechanism 310, thereby providing the caregiver and wearer with the proper orientation of the garment. This makes it easier to put the garment on. Other visible indicia 320 may be provided on disposal fastening mechanism 310 that do not appear until the mechanism 310 is employed to secure the garment 10 in a rolled up configuration, as shown in FIG. 6. The figures illustrate the word “DISCARD” being printed on the mechanism 310, and that it is visible after unfolding disposal fastening mechanism 310, and securing garment 10 in a rolled up configuration. This may help remind the caregiver to discard or dispose of the garment 10. Any other indicia 320 can be used in the present invention, as will be appreciated by those skilled in the art.

Other embodiments, uses, and advantages of the invention will be apparent to those skilled in the art from consideration of the specification and practice of the invention disclosed herein. The specification should be considered exemplary only, and the scope of the invention is accordingly intended to be limited only by the following claims.

What is claimed is:

1. A disposable absorbent garment having a longitudinal dimension and a lateral dimension comprising:
a top sheet;
a back sheet having an outer surface;
an absorbent core at least partially disposed between the top sheet and the back sheet, the top sheet, back sheet, and absorbent core making up a main body of the absorbent garment having longitudinal and lateral centerlines, and being configured to provide a front waist region, a rear waist region, and a crotch region longitudinally disposed there between; and
a disposal fastening mechanism disposed on the outer surface of the back sheet that is capable of maintaining the garment in a rolled up configuration after the garment is rolled up,
wherein the disposal fastening mechanism includes visible indicia disposed thereon.

2. The absorbent article of claim 1, further comprising elastic leg gathers comprising one or more elastic materials disposed adjacent a lateral edge of the crotch region, and standing leg gathers disposed on the top sheet adjacent the lateral edge of the crotch region.

3. The absorbent garment of claim 1, wherein the disposal fastening mechanism is an adhesive tape.

4. The absorbent garment of claim 3, wherein the adhesive tape is elastically extensible.

5. The absorbent garment of claim 1, wherein the disposal fastening mechanism is disposed on the outer surface of the back sheet in a folded configuration such that a first end of the disposal fastening mechanism is permanently adhered to the outer surface of the back sheet, and a second end of the disposal fastening mechanism is temporarily adhered to a folded portion thereof.

6. The absorbent garment of claim 1, wherein the visible indicia comprises words.

7. The absorbent garment of claim 1, wherein the visible indicia comprises a combination of words and graphics.

8. The absorbent garment of claim 1, wherein the visible indicia comprises an insignia indicating the manufacturer or origin of the garment.

9. The absorbent garment of claim 1, further comprising a chassis layer, whereby the main body of the absorbent garment is associated with the chassis layer, and the disposal fastening mechanism is disposed on an outer surface of the chassis layer.

10. The absorbent garment of claim 1, wherein the disposal fastening mechanism is disposed on the outer surface of the back sheet such that it can be extended longitudinally to secure the garment in a rolled up configuration.

11. The absorbent garment of claim 1, wherein the disposal fastening mechanism is disposed on the outer surface of the back sheet such that it can be extended laterally to secure the garment in a rolled up configuration.

12. The absorbent garment of claim 1, further comprising waist elastics disposed in the first and second waist regions.

13. The absorbent garment of claim 12, wherein the waist elastics are disposed between two outer non-woven sheets, which are associated with the main body.

14. A method of making an absorbent article comprising:
a) preparing a top sheet and a back sheet;
b) preparing an absorbent core;
c) disposing the absorbent core at least partially between the top sheet and the back sheet, whereby the top sheet, back sheet, and absorbent core are prepared and arranged such that they form a main body of the absorbent garment having a longitudinal and lateral dimension, and having a first waist region, a second waist region longitudinally opposite the first waist region, and a crotch region disposed between the waist regions;
d) preparing a disposal fastening mechanism having visible indicia disposed thereon, the disposal fastening mechanism being capable of maintaining the garment in a rolled up configuration after the garment is rolled up; and
e) placing the disposal fastening mechanism on an outer surface of the back sheet.
